(19) United States
(12) Patent Application Publication
(10) Pub. No.: US 2008/0312134 A1
(43) Pub. Date: Dec. 18, 2008

(54) Vesicles for active macromolecule delivery

(76) Inventors: Thomas Hirt, Rebstein (CH); Zhihua Lu, John's Creek, GA (US)

Correspondence Address: LAW OFFICE OF COLLEN A. BEARD, LLC, P. O. BOX 1064, DECATUR, GA, 30031-1064, US

(21) Appl. No.: 12/157,042

(22) Filed: Jun. 6, 2008

Related U.S. Application Data
(60) Provisional application No. 60/934,063, filed on Jun. 11, 2007.

(51) Int. Cl. A61K 38/02; A61K 31/7105; A61K 31/713; A61K 47/30; C08G 69/26; C07K 1/04; A61K 48/00; A61K 31/711

(52) U.S. Cl. 514/2; 514/44; 514/772.3; 528/332; 527/200

(57) ABSTRACT

Vesicles for delivery of active macromolecules, such as oligoaminoacids, oligonucleotides, microRNA, siRNA, dsRNA, single stranded oligonucleotides, DNA, peptide nucleic acids, peptides, proteins, and some synthetic organic drugs which are made from amphiphilic segmented copolymers where one segment contains the active macromolecule. In one preferred embodiment, the active macromolecule is contained in the hydrophilic C segment of an amphiphilic ABC segmented copolymer.

### RELATED APPLICATION

The present application is related to and claims priority to U.S. Provisional Application Ser. No. 60/934,063 filed Jun. 11, 2007, the entire contents of which are incorporated herein by reference.

### BACKGROUND OF THE INVENTION

The invention is related to drug delivery and more specifically related to vehicles and methods for delivery of active macromolecules.

Many therapies have been developed using large biologically active macromolecules, such as nucleic acids and proteins. An issue with use of such molecules is getting the molecules to the intended point of use. The cellular uptake of these molecules is poor due to their large size, hydrophilicity, and negatively charged backbone. In addition, they are very susceptible to degradation by nucleases. Oligonucleotides as well as RNA are strongly dependent on carriers to cross cellular membranes, to escape from endosomes, and to maintain their physicochemical properties in extra- and intracellular matrices.

Complexation with polycations has been a method used to encourage intracellular uptake. See Jans et al., Nature Reviews, Genetics, Vol. 6, (April 2005) 299. Cationic liposomes have been studied as one of the most promising non-viral gene delivery systems. However, this method has major drawbacks such as the formation of large aggregates at higher concentrations and the instability in serum due to cationic lipids. Chong-Kook Kim et al., Biomaterials 25 (2004) 5893-5903.

According to the present invention, an active macromolecule is a portion of an amphiphilic segmented copolymer that forms hollow vesicles. An advantage of this method of delivering an active macromolecule is that the active macromolecule does not need to be complexed to a polycation. Thus it can more easily get into the nucleus or be active in the cytoplasm. Another advantage is that sensitive molecules such as oligonucleotides are protected from degradation.

### SUMMARY OF THE INVENTION

The present invention is drug delivery vehicles for delivery of active macromolecules, such as oligoaminoacids, oligonucleotides, microRNA, siRNA, dsRNA, single stranded oligonucleotides, DNA, peptide nucleic acids, peptides, proteins, and some synthetic organic drugs. The vesicles are made from amphiphilic segmented copolymers where one segment contains the active macromolecule. In one preferred embodiment, the active macromolecule is contained in the hydrophilic C segment of an amphiphilic ABC segmented or triblock copolymer.

In a preferred embodiment, the vesicles enter into the cell and deliver the active macromolecule into the cell. In one embodiment, the active macromolecule must be cleaved from the remainder of the amphiphilic polymer to be active but this may not be necessary for all embodiments. Some macromolecules may have their desired activity even if all or a portion of the remainder of the amphiphilic polymer remains attached. Cleavage may be responsive to a particular condition, i.e. pH, may be enzymatic, or may be accomplished through other means.

Most desirably, the copolymer is an ABC segmented copolymer, where A is hydrophilic, B is hydrophobic, and C contains a hydrophilic active macromolecule. A vesicle having hydrophilic inner (C) and outer (A) layers and a middle (B) hydrophobic layer will be formed.

“Hollow particle” and “vesicle” are synonymous and refer to a particle having a hollow core or a core filled with a material to be delivered or released. Vesicles may have a spherical or other shape.

The terms “nanospheres” and “nanocapsules” are used synonymously herein and refer to vesicles that are stabilized through crosslinking. While the nanocapsules are generally in the nanometer size range, they can be as large as about 20 microns. Thus, the term is not limited to capsules in the nanometer size range. The capsules can be spherical in shape or can have any other shape. The terms “microspheres” and “microcapsules” may be used to refer to vesicles or capsules having a size up to about 1000 microns.

The term “polymerization” as used herein refers to end to end attachment of the amphiphilic copolymers.

The term “crosslinking” as used herein refers to interpolymer linking of all types, including end to end attachment (“polymerization”) as well as covalent or ionic bonding of any portion of a copolymer to another copolymer. Crosslinking can be through end groups or internal groups and can be via covalent, ionic, or other types of bonds.

The term “active macromolecule” as used herein refers to a biologically active macromolecule such as an oligoaminoacid, oligonucleotide, microRNA, siRNA, dsRNA, single stranded oligonucleotide, DNA, peptide nucleic acid, peptide, protein, and some synthetic organic drugs.

### DETAILED DESCRIPTION OF THE INVENTION

The invention is drug delivery vehicles comprising vesicles formed from amphiphilic segmented copolymers, where at least one segment includes an active macromolecule.

Vesicles and Amphiphilic Copolymers

The formation of vesicles from amphiphilic copolymers is taught in U.S. Pat. No. 6,916,488 to Meier et al., which is useful as a guide for the invention taught herein. The formation of vesicles from an amphiphilic copolymer is a result of the amphiphilic nature of the copolymer. Aggregation of the copolymers occurs via non-covalent interactions and therefore is reversible, although the vesicles can be crosslinked to provide additional stability. It should be understood that the copolymers can be polymerized via end groups, crosslinked via internal crosslinkable groups, or a combination of end group and internal group polymerization/crosslinking can be used. If the vesicles are crosslinked, the resulting micro or nanocapsules are more stable, shape-persistent, and may preserve their hollow morphology even after they are removed from an aqueous solution.

In a preferred embodiment of the invention, triblock amphiphilic copolymers are used to form vesicles. The copolymers have the structure ABC, where A and C are hydrophilic polymers and B is a hydrophobic polymer. At least one of A, B, and C contains the active macromolecule. Under appropriate conditions, the ABC copolymer will form vesicles having an outer hydrophilic surface. If C is the hydrophilic active macromolecule, it will desirably be on the interior of the vesicle.

While this preferred embodiment is primarily discussed herein, the invention is not limited to this embodiment. Other possible embodiments include an ABA copolymer where A is hydrophilic and contains the active macromolecule; an ABC copolymer, where B contains the active macromolecule (A and C hydrophilic, B hydrophobic); an ABA copolymer where B contains the active macromolecule (A hydrophilic, B hydrophobic); and an ABC copolymer where A contains the active macromolecule and the vesicles form with A on the exterior of the vesicles (A and C hydrophilic, B hydrophobic). In each embodiment, more than one segment could include an active macromolecule.

The stability of a particular vesicle (and the length of time it takes to degrade) depends in a large part on the strength of the hydrophobic and hydrophilic interactions between the copolymers. The strength also depends upon the stability of the junction between the hydrophilic and hydrophobic segments, and the junction between the hydrophilic or hydrophobic segment and the polymerizing unit, if one is used. The stability further depends upon the strength of the polymerization or crosslinking, if such is used. The stability of the vesicles can be decreased by the introduction of weak links, such as biodegradable links or ionic crosslinks, between the hydrophilic and hydrophobic segments, within the hydrophilic or hydrophobic segment, or between the hydrophilic or hydrophobic segment and the polymerizing unit.

Crosslinking can be achieved using many standard techniques, including photopolymerization, for example, of acrylate groups in the presence of a photoinitiator, or through the use of an alkylating agent, polyaddition reaction with diisocyanates, use of carbodiimides with dicarboxylic acids, or complexation with metal ions. Crosslinking can also be achieved using side groups and end groups which can be polymerized by free radical polymerization, side groups which can be polymerized by cationic polymerization, and side groups which can be polymerized by ring-opening polymerization.

In a preferred embodiment, the vesicles are formed from an ABC triblock copolymer, where A and C are hydrophilic and B is hydrophobic, and C includes the active macromolecule. The copolymer, and thus the vesicles, may be degradable. One way to design wholly or partially degradable vesicles is by having the bond between the A and B segments and/or the B and C segments degradable. Another way is to have either or both of A or B degradable. It is particularly desirable that B is biodegradable so that it can be broken down and cleared by the body, however this is not an absolute requirement. Since A is water soluble and below 40,000 g/mol it will be cleared through the kidneys. Since the active macromolecule is contained in the C segment, it would be desirable that the active macromolecule, or C in its entirety, be cleavable from the rest of the copolymer once the vesicle has reached its intended target. This link, between B and C or connecting the active macromolecule to the rest of the copolymer, could be enzymatically degradable (such as by having a disulphide linkage) or hydrolyzable under the conditions in the cell, e.g. pH 5.5 in the endosome. Examples of pH sensitive bonds include ester and phosphoramidate bonds. Other methods of cleaving the active macromolecule, or C could be used.

In addition to the hydrophilic and hydrophobic segments A, B, and C, the copolymers may include additional hydrophobic and/or hydrophilic segments or pendant groups, as well as crosslinkers such as monomers or macromers with reactive groups, surfactants, and crosslinking initiators, especially photoinitiators.

Targeting or biological signal molecules can be attached to the vesicles. The surface of the vesicles can easily be modified with specific targeting ligands. This can be achieved, for example, by copolymerization with a small fraction of ligand-bearing comonomers, e.g. galactosyl-monomers. It is well known that such polymer-bound galactosyl-groups are recognized by the receptors at the surface of hepatocytes (Weigel, et al. J. Biol. Chem. 1979, 254, 10830). Such labeled vesicles will migrate to the target.

In one embodiment, membrane translocation signals (MTS) are attached to the vesicles—either after vesicle formation or by attaching MTS's to some of the A segments of an ABC segmented copolymer before vesicle formation. An MTS will assist in cell entrance via membrane penetration. MTS's are described, for example, in C-H. Tung, et al, Adv. Drug Delivery Reviews 55 (2003) 281; G. Divita et al, Nucleic Acids Research, 2003, Vol. 31, No. 11, 2717-2724; and M. R. Eccles et al., FEBS Letters 558 (2004) 63-68.

In addition to the following guidance for the selection of the hydrophilic and hydrophobic segments, selection of the polymers, molecular weights, and other aspects of the hydrophobic and hydrophilic segments is covered in U.S. Pat. No. 6,916,488 to Meier et al. and one skilled in the art can look there and elsewhere for guidance. Preparation of the copolymers and the vesicles is also taught in the Meier patent and one skilled in the art can use the teachings therein as a guide to make the copolymers and vesicles.

The mean molecular weight of segment A is desirably in the range from about 1000 to about 40,000, preferably in the range from about 2000 to 20,000. B desirably has a molecular weight of about 2000 to 20,000, preferably between about 3000 and 12,000. C is desirably about 200 to 40,000, preferably between about 200 and 20,000. A should be equal to or larger than C.

Desirably, to facilitate entry into the cell, the vesicles range from about 10 to 1500 nm in diameter, more preferably about 20 nm to about 600 nanometers in diameter. Of course if the vesicles do not need to enter the cell they can be larger in size.

Segment A

The segment A includes at least one hydrophilic polymer, such as, but not limited to, polyoxazoline, polyethylene glycol, polyethylene oxide, polyvinyl alcohol, polyvinylpyrrolidone, polyacrylamide, poly(meth)acrylic acid, polyethylene oxide-co-polypropyleneoxide block copolymers, poly (vinylether), poly(N,N-dimethylacrylamide), polyacrylic acid, polyacyl alkylene imine, polyhydroxyalkylacrylates such as hydroxyethyl methacrylate (HEMA), hydroxyethyl acrylate, and hydroxypropyl acrylate, polyols, and copolymeric mixtures of two or more of the above mentioned polymers, natural polymers such as polysaccharides and polypeptides, and copolymers thereof, and polyionic molecules such as polyallylammonium, polyethyleneimine, polyvinylbenzyltrimethylammonium, polyaniline, sulfonated polyaniline, polypyrrole, and polypyridinium, polythiophene-acetic acids, polystyrenesulfonic acids, zwitterionic molecules, and salts and copolymers thereof.

The hydrophilic segment A preferably contains a predominant amount of hydrophilic monomers. A hydrophilic monomer is a monomer that typically gives a homopolymer that is soluble in water or can absorb at least 10% by weight of water.

The hydrophilic polymer A can be selected for stealth properties or tissue adhesion depending on the delivery route for which it is designed (IV, mucosal, etc.). Examples include polyethyleneglycol (PEG), poly (2-methyloxazoline) (PMOXA), polyaminoacids, polyacrylic acid, thiolates, and chitosans. In one embodiment, segment A is a polysaccharide which also acts as a targeting ligand.

Segment B

The amphiphilic segmented copolymer includes at least one segment B that includes a hydrophobic polymer. Any of a number of hydrophobic polymers can be used, such as, but not limited to, polysiloxanes such as polydimethylsiloxane and polydiphenylsiloxane, perfluoropolyether, polystyrene, polyoxypropylene, polyvinylacetate, polyoxybutylene, polyisoprene, polybutadiene, polyvinylchloride, polyalkylacrylate (PAA), polyalkylmethacrylate, polyacrylonitrile, polypropylene, PTHF, polymethacrylates, polyacrylates, polysulfones, polyvinylethers, and poly(propylene oxide), and copolymers thereof.

The hydrophobic segment B preferably contains a predominant amount of hydrophobic monomers. A hydrophobic monomer is a monomer that typically gives a homopolymer that is insoluble in water and can absorb less than 10% by weight of water.

In one embodiment, segment B includes a fusogenic molecule or segment (e.g. hydrophobic domain of the peptide transportan) which assists in penetration of the endosomal membrane and escape from the endosome.

Section B could also include a hydrophobic active macromolecule, in which case B should have a molecular weight of at least 2000 g/mol.

Segment C and the Active Macromolecule

The segment C is hydrophilic, to enable formation of the vesicle, but also contains an active macromolecule. The entire segment C can be the active macromolecule, or the active macromolecule can be part of the segment.

The active macromolecule can be an oligoaminoacid, oligonucleotide, microRNA, siRNA, dsRNA, single stranded oligonucleotide, DNA, peptide nucleic acid, peptide, protein, and some synthetic organic drugs. Potentially suitable synthetic organic drugs include those that are water soluble and have a molecular weight of at least about 200. Candidates include antibiotics (e.g. Linezolid), pain killers (e.g. hydromorphone), or chemotherapeutics (e.g. doxorubicin).

Preferably, the active macromolecule is hydrophilic but hydrophobic macromolecules can also be used if they are in the B segment. It may also be possible that the active macromolecule itself is not hydrophilic or hydrophobic so long as the segment C, or B, is hydrophilic, or hydrophobic in the case of B, in its entirety.

Method for Delivering an Active Macromolecule

The methods for delivering an active macromolecule include the first step of forming an amphiphilic segmented copolymer including the active macromolecule in one segment. In the preferred embodiment, the active macromolecule is part of the C segment of an ABC copolymer, where A and C are hydrophilic and B is hydrophobic. Using appropriate A, B, and C segments, as described above, and conditions, also as described above and in U.S. Pat. No. 6,916,488 to Meier et al., vesicles can be formed having A on the exterior and C on the interior. In this way the active macromolecule is protected.

In general, vesicles can be made by a number of means known to those skilled in the art. Self assembly techniques are preferred. In one embodiment, the amphiphilic copolymer is dissolved in a solvent such as ethanol at a concentration of from about 5% to 30%. The polymer solution is then added to an aqueous solution, with stirring. This procedure generally leads to a dispersion of segmented copolymer vesicles of a rather broad size distribution. The size distribution can be controlled by methods known to those skilled in the art of preparing vesicles. In addition, the size distribution can be selected by passing the polydisperse vesicles through one or more filters having a defined pore size. The resulting vesicle dimensions are directly determined by the pore diameter of the filter membrane.

The vesicles can be used as experimental, diagnostic, and therapeutic reagents. For therapeutic usage, the vesicles can be administered orally, by injection or by pulmonary, mucosal, or transdermal routes. The vesicles will usually be administered in combination with a pharmaceutically acceptable carrier. Pharmaceutical carriers are known to those skilled in the art. The appropriate carrier will typically be selected based on the mode of administration. Pharmaceutical compositions may also include one or more active ingredients such as antimicrobial agents, anti-inflammatory agents, and analgesics.

Dosing is dependent on severity and responsiveness of the condition to be treated, but will normally be one or more doses per day, with course of treatment lasting from several days to several months or until the attending physician determines no further benefit will be obtained. Persons of ordinary skill can determine optimum dosages, dosing methodologies, and repetition rates.

Once the vesicles are administered, they can enter the cell, if so designed and segment C or the active macromolecule portion thereof is cleaved from the remainder of the copolymer. The active macromolecule is thus able to express its desired activity. Desirably, segments A and B are cleaved and B is biodegradable. Since A is water soluble and has a molecular weight less than 40,000 it will be cleared from the body.

### EXAMPLES

The examples below serve to further illustrate the invention, to provide those of ordinary skill in the art with a complete disclosure and description of how the compounds, compositions, articles, devices, and/or methods claimed herein are made or could be made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

Materials and Equipment: All reagents, solvents were of reagent grade and were purchased from commercial sources such as Polysciences, Fluka, Aldrich and Sigma.

General Analysis: The polymers synthesized according to these examples can be chemically analyzed using structure-determining methods such as nuclear (proton and carbon-13) magnetic resonance spectroscopy, infrared spectroscopy and UV-visible spectroscopy. Polymer molecular weights can be determined using gel permeation chromatography. Aqueous solution properties such as micelle and vesicle formation can be determined using fluorescence spectroscopy, UV-visible spectroscopy, and laser light scattering instruments.

### Example 1

### PEG5000-PDMS5000-[Met-OMe11]-Substance P1350 Copolymer and Vesicles

This is an example of an ABC copolymer, wherein A is a hydrophilic polymer, B is a hydrophobic polymer, and C is a peptide [Met-OMe11]-Substance P (Mw=1363 g/mol).

Synthesis of PEG5000-PDMS5000-OH

The synthesis of this material is modified from a method described by R. Stoenescua and W. Meier, Chem. Commun. 2002, 3016. A solution of 15.25 g of poly(ethylene glycol) ethyl ether (Mn=5 000 g/mol) in dry tetrahydrofuran ([PEG]0=125 g/L) is added to a dispersion of potassium hydride in THF. 18-crown-6 (19 mg, 7.11 3 1025 mol) is added and the reaction solution stirred for 4 h. Subsequently, 1.2 ml of 2,6-dimethylpyridine is added dropwise and the reaction stirred for another 30 min. The resulting alcoholate anion is used as an initiator for the anionic ring opening polymerization of octamethyltetracyclosiloxane (D4) (18.7 g, 63 mM). The polymerization time is 20 h at a temperature of 55 C. The chain growth is terminated using methacryloyloxypropyldimethylchlorosilane (2.8 g, 12.6 mM). The resulting PEG-PDMS diblock copolymer is purified by dialfiltration through a membrane (Mn 1000 cutoff) in water/ethanol. After evaporation of the solvent the pure PEG-PDMS diblock copolymer is obtained. Reduction of the ester end-group is accomplished using a modified Bouveault-Blanc method. An excess (40%) of sodium in ethanol is added to a solution of the polymer in ethanol (1+1, v/v) and allowed to react for 16 h at 75 C. The solvent is removed under reduced pressure.

The polymer is purified by dialfiltration in water through a membrane (Mn 1000 cutoff). After excess solvent is removed under reduced pressure, the polymer is dried under vacuum.

Addition of [Met-OMe11]-Substance P1350

The functionalized copolymer (10 g) obtained as above is dried under vacuum and then reacted with 1.0 molar equivalent of succinic anhydride in dry chloroform at 65 C for 24 h. The resulting PEG-PDMS-COOH is reacted with 1.05 molar equivalents of peptide [Met-OMe11]-Substance P (Mw=1363 g/mol) and 1.5 molar equivalents of 1,3-dicyclohexylcarbodiimide (DCC) in dimethyl sulfoxide at room temperature for 24 h. When the reaction is complete, the mixture is filtered and solvent is removed under reduced pressure. The product is purified by dialfiltration through a membrane (Mn 1000 cutoff) in water/ethanol. After solvent is removed under reduced pressure, the polymer is dried under vacuum and stored at −20 C.

Formation of Vesicles

The resulting copolymer (50 mg) is stirred with 5 ml of PBS water for 24 h. The solution is extruded through 0.45 um and 0.2 um filters and the received solution is kept at 4 C. For further purification, the received solution is cleaned over a Sepharose® 4B column and the first opaque fraction is collected and kept at 4 C.

### Example 2

### PEG1000-PCL5000-Active Macromolecules and Vesicles Using Ester-Amide Linkage

This is an example of an ABC copolymer, wherein A is a hydrophilic polymer, B is a biodegradable hydrophobic polymer, and C is an active macromolecule that can be attached via an amino group.

Preparation of PEG1000-PCL5000

Poly(ethylene glycol) methyl ether (Mn 5000 g/mol, 10 g) is dried under vacuum at 100 C for 12 h and dissolved in 50 ml of dry chlorobenzene. 10 g of caprolactone and 50 ul of stannous 2-ethylhexanoate are added. The solution is heated at 120 C for 20 h. The polymer solution is cooled and then purified by several precipitations from chloroform to diethyl ether. The copolymer is dried under vacuum.

Preparation of PEG5000-PCL5000-succinate

1.2 molar equivalents of succinic anhydride is added to a solution of 10 g of PEG5000-PCL5000 (made as described above) in 100 ml dry chloroform. The mixture is refluxed under a nitrogen atmosphere for 24 h. The solution is cooled and added to 1000 ml hexane to precipitate the carboxyl terminated polymer. It is further purified by repeated (3 times) precipitation from chloroform to hexane. The polymer is dried under vacuum at 40 C. This polymer is immediately used to prepare copolymer with activated terminal group PEG-PCL-NHS as described below.

Preparation of PEG5000-PCL5000-NHS

PEG-PCL-succinate copolymer (10 g) is dissolved in 100 ml dry DMF. The solution is cooled in ice/water bath and 1.5 molar equivalents of 1,3-dicyclohexylcarbodiimide (DCC) and 1.05 molar equivalents of N-hydroxysuccinimide (NHS) are added to the reaction mixture. The mixture is stirred in ice/water bath for 6 h and then stirred overnight at room temperature. Dicyclohexylurea is removed by filtration or centrifugation. After removal of solvent under vacuum the product is repeatedly precipitated from chloroform to hexane. The product is vacuum dried.

Addition of Active Macromolecule

Functionalized diblock polymer PEG5000-PCL5000-NHS can be reacted with an amino-containing drug or with the amino group of a peptide or protein, DNA, or SiRNA. For example, PEG5000-PCL5000-NHS (10 g) prepared as above is dissolved in 50 ml of dry DMF and added to a stirred solution of peptide (0.5-0.95 molar equivalents of PEG-PCL-NHS) in DMF. The reaction is carried out in the presence of triethylamine at 25 C for 24 h. The solvent is removed under vacuum and the product purified by diafiltration in water/ethanol. After solvent is removed by vacuum, the product is kept at −80 C.

Addition of Oxytocin to Make PEG5000-PCL5000-Oxytocin

PEG5000-PCL5000-NHS (10 g) prepared as above, in 50 ml of dry DMF is added to a stirred solution of oxytocin (0.5-0.95 molar equivalents of PEG-PCL-NHS) in DMF. The reaction is carried out in the presence of triethylamine at 25 C for 24 h. The solvent is removed under vacuum and the product purified by diafiltration in water/ethanol. After solvent is removed by vacuum, the product is kept at −20 C.

Formation of Vesicles

The resulting ABC triblock copolymer can be further dispersed in aqueous solution to form vesicles. 50 mg of the block copolymer is dissolved in 0.5 ml of EtOH by heating the solution above 60 C for 1-3 minutes. The solution that has formed is slowly dropped into 10 ml of distilled water under stirring. Afterwards the solution is extruded through 0.45 um and 0.2 um filters. The received vesicle solution is stored at 4 C.

### Example 3

### PEG1000-PCL5000-Active Macromolecules and Vesicles Using Michael Addition

This is an example of an ABC copolymer, wherein A is a hydrophilic polymer, B is a biodegradable hydrophobic polymer, and C is an active macromolecule that can be attached using Michael addition.

Preparation of PEG5000-PCL5000-acrylate

PEG5000-PCL5000 copolymer (10 g) made as described in Example 2 is dissolved in dry methylene chloride. The solution is kept at 0-5 C. Equivalent molar acryloyl chloride in methylene chloride is dropwise added in the solution in the present of pyridine. The mixture is warmed to room temperature and filtered. The polymer is purified by repeated precipitation from methylene chloride to diethyl ether and dried under vacuum.

Addition of siRNA

The PEG5000-PCL5000-acrylate is mixed with multi-thiol-modified SiRNA [1/10 molar equivalent, HS-(CH2)n-SiRNA] in ethanol. The Michael reaction is carried out in the dark for 48 h with or without a catalyst. After removal of solvent under vacuum, the product is purified by diafiltration through membrane (Mw cutoff 5 kDa in water/ethanol and then freeze-dried to obtain PEG-PCL-SiRNA. See J. Hubbell et al., Journal of Controlled Release 102 (2005) 619-627; K. Kataoka et al., Biomacromolecules, 2005, 6, 2449-2454.

Formation of Vesicles

The copolymer powder (50 mg) is stirred with 5 ml of 2 mM PBS solution for 24 h. Afterwards the solution is extruded through 0.45 um and 0.2 um filters. The received vesicle solution is stored at 4 C.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A drug delivery vehicle for delivery of an active macromolecule comprising vesicles made from amphiphilic segmented copolymers having an ABA or ABC structure, wherein segments A and C are each hydrophilic, segment B is hydrophobic, and one segment comprises the active macromolecule.

2. The drug delivery vehicle of claim 1 wherein the amphiphilic segmented copolymer is an ABC copolymer and segment C includes a hydrophilic active macromolecule.

3. The drug delivery vehicle of claim 1 wherein the active macromolecule is selected from the group consisting of oligoaminoacid, oligonucleotide, microRNA, siRNA, dsRNA, single stranded oligonucleotide, DNA, peptide nucleic acid, peptide, protein, and some synthetic organic drugs.

4. The drug delivery vehicle of claim 2 wherein all or a portion of segment C is cleaved from the remainder of the amphiphilic segmented copolymer in vivo.

5. The drug delivery vehicle of claim 4 wherein the cleavage is via hydrolysis or enzymatic action at the site of intended activity.

6. The drug delivery vehicle of claim 1 wherein the amphiphilic segmented copolymer is biodegradable.

7. The drug delivery vehicle of claim 1 wherein the link between segment A and segment B or segment B and segment C is biodegradable and segment B is biodegradable.

8. The drug delivery vehicle of claim 1 wherein segment B is biodegradable.

9. The drug delivery vehicle of claim 1 wherein segment A has a molecular weight between about 1000 to 40,000, segment B has a molecular weight between about 2000 to 20,000, and segment C has a molecular weight between about 200 and 40,000.

10. The drug delivery vehicle of claim 9 wherein segment A has a molecular weight between about 2000 to 20,000, segment B has a molecular weight between about 3000 to 12,000, and segment C has a molecular weight between about 200 and 20,000.

11. The drug delivery vehicle of claim 1 wherein the vesicles range from about 10 to 1500 nm in diameter.

12. The drug delivery vehicle of claim 1 wherein segment B includes a hydrophobic active macromolecule.

13. The drug delivery vehicle of claim 1 wherein segment A includes the hydrophilic active macromolecule.

14. The drug delivery vehicle of claim 1 where a targeting ligand is attached to segment A.

15. An amphiphilic segmented copolymer for forming drug delivery vesicles for delivering an active macromolecule, having an ABA or ABC structure, wherein segments A and C are each hydrophilic, segment B is hydrophobic, and one segment comprises the active macromolecule.

16. The amphiphilic segmented copolymer of claim 15 having an ABC structure and segment C includes a hydrophilic active macromolecule.

17. The amphiphilic segmented copolymer of claim 15 wherein the active macromolecule is selected from the group consisting of oligoaminoacid, oligonucleotide, microRNA, siRNA, dsRNA, single stranded oligonucleotide, DNA, peptide nucleic acid, peptide, protein, and some synthetic organic drugs.

18. The amphiphilic segmented copolymer of claim 15 wherein all or a portion of segment C can be cleaved from the remainder of the amphiphilic segmented copolymer via hydrolysis or enzymatic action at the site of intended activity.

19. The amphiphilic segmented copolymer of claim 15 wherein the link between segment A and segment B or segment B and segment C is biodegradable and segment B is biodegradable.

20. The amphiphilic segmented copolymer of claim 15 wherein segment B is biodegradable.

21. The amphiphilic segmented copolymer of claim 15 wherein segment A has a molecular weight between about 1000 to 40,000, segment B has a molecular weight between about 2000 to 20,000, and segment C has a molecular weight between about 200 and 40,000.

22. The amphiphilic segmented copolymer of claim 15 where segment B includes a hydrophobic active macromolecule.

23. The amphiphilic segmented copolymer of claim 15 where segment A includes the hydrophilic active macromolecule.
